(19) United States
(12) Patent Application Publication
(10) Pub. No.: US 2026/0240609 A1
(43) Pub. Date: Aug. 20, 2026

(54) MICROROBOTIC UNIT FOR PROPULSION MOVEMENT AND POSITIONING IN ORGANIC CAVITIES

(71) Applicants: Andreas KARGUTH, Tüttleben (DE); FESTO SE & CO. KG, Esslingen (DE)

(72) Inventor: Andreas KARGUTH, Tüttleben (DE)

(21) Appl. No.: 18/992,692

(22) PCT Filed: Jul. 7, 2023
(86) PCT No.: PCT/EP2023/068941

§ 371 Date: Jan. 9, 2025

(30) Foreign Application Priority Data
Jul. 12, 2022 (DE) 10 2022 117 389.5

(51) Int. Cl. A61B 34/30; A61M 25/01; A61M 25/10

(52) U.S. Cl. CPC A61B 34/30; A61B 2034/302; A61M 25/0116; A61M 25/0158; A61M 25/10

(57) ABSTRACT

A microrobotic unit for propulsion movement and positioning in organic cavities comprises two spreadable modules and a pushing module which is located between them; and a supply connection at the rear spreadable module, which is used to supply a supply medium. Each spreadable module has at least one outer balloon envelope, which expands when the supply medium is supplied a surface structure is provided on the outside of the balloon envelope and causes the balloon element to be anchored in surrounding tissue. The pushing module has a bellows structure causing an axial expansion when the supply medium is supplied and an axial contraction when the supply medium is removed. The bellows structure has an outer support structure to prevent or limit a change in its dimensions in the radial direction. The axially extending support structure is in the form of a plurality of toggle lever structures arranged in a row.

### BACKGROUND OF THE INVENTION

The present invention relates to a microrobotic unit for propulsion movement and positioning in organic cavities. This unit comprises at least one front spreadable module and one rear spreadable module, a pushing module which is arranged between the two spreadable modules and connected to them, and a supply connection which is axially connected to the rear spreadable module and serves to supply at least one, preferably several supply media.

In various areas of technology there is a need to penetrate into hard-to-reach cavities with miniaturized technical units for exploration and/or to carry out repairs or the like. Likewise, in medical applications there is a need to penetrate or navigate between soft (compliant) tissue structures, especially in the so-called organ folds. For example, there are externally actuated endoscopes for such tasks, which can be used to examine the interior of organisms or technical cavities and to manipulate structures. Rigid and flexible endoscopes are known, which are equipped with a wide variety of tools and optical units. In human medicine, endoscopes are used for diagnostics as well as for minimally invasive surgical procedures.

In recent times, new diagnostic and therapeutic methods have required technical carrier systems with the help of which tissue samples from organs in the peritoneal cavity can be taken, special sensors for in-vivo tissue analysis can be positioned, personalized medications can be precisely placed or injected into damaged organs, drains can be placed or other tissue manipulations can be carried out.

The solutions available to date are either based on conventional surgical methods or use technical assistance systems for minimally invasive surgery (MIC). These are either manually guided laparoscopy instruments (tools and camera) or instruments that are guided by a robotic carrier system (e.g., DaVinci robot system). In both MIC procedures, the patient's abdominal cavity must be filled with large volumes of CO₂ and inflated to make room for instrument manipulation. This places a considerable burden on the patient.

Another access option is the use of special catheter systems, the tips of which must be advanced and steered manually (directly or with the assistance of a drive system) by the surgeon. This technique provides access via body openings and/or vessels and instrument navigation is controlled by imaging techniques. Navigation in the peritoneal space between the organs and between the connective tissue layers is difficult with this method and not possible in certain areas. In the known technical support systems, the drive systems for generating movement are arranged externally and the effort required for safe movement transmission is high.

WO 2018/154326 A1 shows a robotic device which consists of several segments covered by elastic material. The segments allow for contraction and lengthening, bending, and forward and backward movement.

A maintenance and movement system for a microrobot for intravascular therapy is known from KR 101009053 B1. The system includes movement and holding elements as well as elements for moving the microrobot.

U.S. Pat. No. 6,702,734 B2 describes a self-driving endoscopic microrobot and a system for intestinal endoscopy. The microrobot includes arched support arms, which serve as a housing for protection. The support arms are made of a flexible material.

WO 2021/163615 A1 describes a microrobotic system in hose form, the flexibility of which can be controlled by a medium, wherein the medium is guided in longitudinal channels. The described robotic system includes a robotic surgical tool and a steering system configured to control the robotic surgical tool based on motion angle commands along the X and Y axes. A computing device executing an artificial intelligence program is configured to calculate motion angle commands based on a current position of the robotic surgical tool.

US 2009/0093675 A1 describes a medical system for access via a body opening. The system comprises a medical instrument and an overtube. The overtube defines an overtube lumen that is sized to accommodate the medical instrument. The overtube has a distal end with a tapered portion and a plurality of longitudinally extending slits forming a plurality of flaps. The tapered portion of the overtube is sized relative to the medical instrument such that displacement of the medical instrument through the distal end of the overtube forces the plurality of flaps to move radially outward.

WO 2021/207574 A1 shows an endoscope coupling device with a proximal end configured for attachment to a working end of an endoscope and a visualization portion that enables viewing of tissue. The coupling device further comprises an instrument channel having an open distal end and a proximal end configured for alignment with a working channel of the endoscope. The coupling device is configured to retain and/or secure an instrument that has traversed the instrument channel to ensure that the instrument remains at the target site and/or to facilitate instrument replacement.

DE 10 2020 107 309 A1 relates to a carrier system comprising a central tube with a proximal and a distal end, a receptacle arranged within the central tube and at least one expandable fixing element arranged on the outside of the central tube, which can assume a contracted and an expanded state. The central tube is sealed fluid-tight at at least one end by a closing element. A passage remains between the fixing element and the central tube even when the fixing element is expanded.

US 2019/0159916 A1 describes a device for insertion into a vessel, which is suitable for carrying out a translational movement through the vessel. The device comprises a flexible tube containing a translation actuator. Anchoring actuators are arranged along the length of the flexible tube and are configured to controllably expand against the inner wall of the vessel in coordination with the expansion and contraction of the translation actuator to effect translational motion.

U.S. Pat. No. 5,662,587 concerns a robot for performing endoscopic procedures in flexible and curved human or animal lumens. For this purpose, a large number of segments are connected to each other. Tension segments enclose the lumen walls, other segments contain actuators that cause the endoscope to locally change its shape by bending, stretching, or a combination of bending and stretching. The segments can be controlled to allow worm-or snake-like propulsion movement through a curved and flexible lumen. A pressure gas line attached to the posterior segment delivers compressed gas to insufflate the lumen and can optionally be used to drive the actuators that control the operation of the endoscope segments. The guide segment may include television cameras, ultrasound transducers, biopsy arms, drug delivery systems or other sensors, diagnostic aids, therapeutic devices and surgical tools. Medical instruments and sensors can also be housed in the rear or middle segments.

From DE 102 09 986 B4 an endoscope shaft is known with a movable distal end section which can be bent by means of an actuating device. The distal end section has a decentrally arranged, longitudinally extending hose element which is formed in one piece from a plurality of bellows arranged directly above one another and thereby forms a continuous pressure chamber in order to expand in the longitudinal direction when pressure is applied.

US 2011/0054253 A1 describes a device for the automated translational movement of an endoscope through the digestive tract. The device comprises pneumatic translational movement means and electronic control means which enable the automated and coordinated movement of the translational movement means. The means for translational movement comprise: a radial chamber fixed on the endoscope; a bellows-shaped axial chamber connected at one end to the body of the endoscope; a radial chamber floating on the endoscope and fixed to the second end of the bellows-shaped axial chamber.

U.S. Pat. No. 4,176,662 shows an endoscope with a drive mechanism and at least one transmitter at the distal end that transmits energy wave bursts for tracking the position of the distal end through the use of two or more transducers on the anterior or lateral surfaces of a patient. The drive mechanism may consist of two radially expandable bladders separated by an axially expandable bellows, with only the front bladder attached to the distal end so that expansion and contraction of the bladders in the correct sequence achieves propulsion of the endoscope. However, it has been found that the expandable bellows described not only changes its dimensions axially when a medium is supplied or removed, but also its radial dimension, which leads to an obstruction of the propulsion movement. Another disadvantage is that the radially expandable bubbles show only a slight reduction in their circumference when contracted, so that they are responsible for undesirably high friction forces during the desired axial advancement of the endoscope.

### SUMMARY OF THE INVENTION

Based on the prior art, it is an object of the present invention to provide an improved microrobotic unit that can be moved in a propelled manner and positioned in technical and in particular in organic cavities or organ folds. This unit is intended to allow easy and safe navigation in cavities, especially in the peritoneal space between the organs located there. The unit should also be able to be equipped with various tools, sensors and/or observation elements in order to transport them to a predetermined location. In particular, the movement of the unit in a compliant environment with slippery, sensitive surface layers should be enabled without damaging these surrounding layers, so that tissue injury in organic cavities is largely avoided. The unit should also be designed in such a way that even in the event of a fault, for example in the event of an accidental leakage of a supply medium, there is no risk of danger or damage to the surrounding tissue areas.

This object is achieved by a microrobotic unit according to the appended claim 1. Particular embodiments of the unit according to the invention are mentioned in the following description and in the dependent claims.

The microrobotic unit according to the invention is characterized firstly by the fact that the front and the rear spreadable module each have an outer balloon envelope, the extent of which expands when the supply medium is supplied and contracts when the supply medium is removed. The resulting circumference of the balloon envelope is controlled by the pressure or negative pressure generated within it. Preferably, the modules of the microrobotic unit have a circular cross-section and an axial extension, so that the unit as a whole has a cylindrical shape, wherein the diameter of the cylinder is preferably in the range of 0.5 to 3 cm and the total length is in the range of 1 to 10 cm.

A surface structure is provided on the outside of each balloon envelope of the spreadable modules, which surface structure, when expanded, provides temporary anchoring/fixation in the surrounding tissue without causing any damage to the tissue. According to preferred embodiments, the balloon envelope can be designed with different structured surfaces and selected depending on the application scenario. For example, nubs or bristles can be attached to the surface. Likewise, structure-forming elements can be placed on the initially smooth surface of the balloon envelope.

The pushing module of the microrobotic unit, located between the spreadable modules, has a bellows structure which causes an axial expansion of the pushing module when the supply medium is supplied and an axial contraction of the pushing module when the supply medium is removed. The bellows structure has an outer, axially extending support structure which allows a change in the expansion of the bellows structure in the axial direction, while at the same time limiting or completely preventing a change in the expansion in the radial direction. The axially extending support structure is shaped like several toggle lever structures arranged in a row, which can be integrated into the wall of the bellows structure or can be placed on top. This special bellows structure achieves an energy-optimized feed.

The support structure can, for example, be created by suitably varying material distribution in the bellows structure in order to achieve the toggle lever effect. Alternatively or additionally, the support structure can be reinforced and optimized by stiffening the outer rings of the bellows (e.g., partial, rigid coating). When the bellows is pressurized using the supplied supply medium, there is practically no expansion in the bellows diameter. Since negative pressure is preferably generated in the bellows for the radial contraction thereof, the concrete support structure can still prevent the bellows from “collapsing”.

Preferably, the microrobotic unit comprises several control elements which control the supply and removal of the supply medium to the individual spreadable modules and to the pushing module as a function of control signals. The controls can be located in the unit itself or remotely from it in the area of the supply medium supply.

The main advantages of this unit according to the invention are that it enables very flexible navigation of the unit in the peritoneal space between the organs. The supply and removal of the supply medium allows the unit to be fixed at any location, a forward movement to be generated, as well as a change of direction and a backward movement, each in an unstructured, compliant environment. This allows flexible maneuvering in a yielding environment with slippery surface layers. This also makes it possible to bypass tissue obstacles in the peritoneal cavity and avoid damage to organs, vessels, nerve strands, etc. The position and orientation of the unit can be controlled with little equipment outlay. Since the microrobotic unit itself is actuated for movement, there is no need for a complex flexible rod mechanism to drive it, thereby also protecting the surrounding tissue.

The toggle lever principle applied to the bellows achieves a particularly good ratio of pressure in the bellows chamber to the realized feed, so that the required feed is achieved with only small pressure changes. This also serves to improve operational safety when using the microrobotic unit, since even in the event of a fault, e.g., if the unit is damaged, there is no risk of injury to the surrounding organs or the like due to supply media under high pressure.

The feed and steering of the microrobotic unit are carried out indirectly by an alternating, steerable pushing movement, which can build up pushing forces in both directions and is generated by the pushing module. Depending on the design, the supply medium can be liquid or gaseous, resulting in a hydraulic or pneumatic power transmission system. It goes without saying that supply lines, control elements, etc. are adapted to the selected supply medium. The actual pushing in the axial direction of the unit is generated by the pushing module, which is arranged between the spreadable modules. The spreadable modules are used for temporary fixation by hydraulically or pneumatically expanding their respective balloon envelope, i.e., significantly increasing their circumference, for example by a factor of 1.5 to 3. The feed force generated by the pushing module acts against such a temporary fixation of one of the spreadable modules, so that, depending on the choice of the fixed front or rear spreadable module, a movement axially forward or backward results.

According to a preferred embodiment, the unit comprises a central channel extending from the supply connection through the rear spreadable module and the pushing module to the front spreadable module. In addition, several additional channels are preferably provided, via which all modules are supplied individually or jointly with the supply medium, preferably controlled via the control elements.

Particularly preferably, the front end of the central channel is closed by a flap mechanism so that media or tools can be guided out of the central channel or external materials can be drawn into the central channel. For example, probes, tools and sensors can be guided through the central channel to the tip of the microrobotic unit. An electrically, hydraulically or pneumatically switchable locking mechanism can preferably be present in the central channel to temporarily fix catheter tools and probes. Further channels may be provided, for example a channel for supplying the hydraulic or pneumatic medium (supply medium) and a channel for supplying flushing fluid and/or for draining corresponding fluids.

In an advantageous embodiment, a frustoconical mandrel is mounted axially in front of the front spreadable module in the pushing direction of the unit. This mandrel particularly preferably has a central opening through which additional tools/instruments can be pushed out of the unit, for example blades, clamps or sensors. When the unit is advanced in the organ folds or between individual organs through the surrounding connective tissue, existing connective tissue fibers must be penetrated by the unit. This is done using the method of blunt preparation, i.e., tearing or pushing apart the tissue fibers, comparable to the method used in conventional surgical techniques. This is made possible by the blunt spike at the front end of the unit.

The microrobotic unit is inserted, for example, through a small surgically created opening in the abdominal wall. Based on existing 3D data from the peritoneal cavity obtained by CT, MRI or ultrasound, the desired path to the target point can be calculated, preferably using systems based on artificial intelligence (AI). The movement of the microrobotic unit can preferably be controlled and tracked using a referenced external 3D ultrasound measuring system.

According to an advantageous embodiment, the microrobotic unit comprises one or more sensors, which are, for example, integrated in one of the spreadable modules or arranged in coupled modules. According to a further developed embodiment, the microrobotic unit has a coupled hose system which is connected to the supply connection. The unit can be supplied with electrical energy and/or with the pneumatic or hydraulic supply medium via this hose system. Furthermore, diagnostic or therapy probes can be fed through the hose system or liquid/gaseous media can be added or removed.

According to further preferred embodiments, the microrobotic unit forms a mobile carrier platform for sensors (e.g., for in-situ tissue analysis) or micro-tools for sampling as well as special tools for influencing tissue locally (ablation tools, discontinuing medications or placement of electronic capsules, e.g., to influence the body's own signal lines). Depending on the application, the unit can be equipped with different sensors or tools or the like.

A modified embodiment is distinguished in that several microrobotic units are supplied via a common hose system and are thus coupled to one another. It is also possible for a unit to comprise more than two spreadable modules and/or more than one pushing module. A further modified embodiment uses several, for example three or four, units connected in parallel, each of which is supplied with a separate hose system.

Since the spreading and pushing modules can be activated in alternating sequence via the control elements via the supply medium, many different operating states can be set. This makes it possible to move the entire unit forwards in a feed direction but also backwards again in order to guide the unit out of the cavity again after completion of a treatment procedure. The backward movement can be supported by pulling on the hose system. The unit can also be temporarily fixed at a treatment site by expanding both spreadable modules.

An advantageous embodiment of the unit has additional modules that are coupled axially behind the rear spreadable module. These additional modules can, for example, be storage modules for medication or for supply energy.

An embodiment is advantageous which comprises a camera which is suitable for observing the environment of the microrobotic unit. For example, the camera can be mounted on the frustoconical mandrel to provide optical images of the area in front of the unit.

According to a further developed embodiment, the unit can comprise other sensors for detecting the environmental and contact parameters as well as sensors for detecting the position and movement states.

In a modified embodiment, the supply medium is not supplied via the hose system, but instead electrical energy is supplied to the unit. In this case, the unit comprises one or more pumps which transport a supply medium to the individual modules within the unit, for example to expand the spreadable modules. A combination of internal and externally supplied supply medium is also possible.

According to one embodiment, the bellows structure comprises a grid structure made of plastic or metal as a support structure, which is embedded in a preferably elastic plastic sheath (e.g., made of silicone). The grid structure can preferably be formed as a so-called Nitinol structure (nickel-titanium alloy) or a comparable shape memory alloy.

The described design of the pushing module and its components enables the pressure built up in the bellows structure to be optimally converted into a feed movement without energy losses for the actuation of the inherent structure (e.g., overcoming intrinsic elasticities). In particular, an increasing internal pressure in the pushing module causes a force effect both on the effective surfaces in the pushing direction and on the enclosing wall surfaces through the aforementioned toggle lever principle, so that a movement and force effect is generated in the pushing direction. As an alternative to the toggle lever principle, the principle of the so-called Nuremberg scissors (joint chain made up of several crossed rods) can also be used in modified versions. Preferably, the outer diameter of the bellows structure remains constant when pressure is applied (from the inside) or is not exceeded beyond a predetermined value.

According to a preferred embodiment, the central bellows element of the bellows structure can be surrounded or shielded by an outer coil spring or other longitudinally elastic structures. The outer coil spring extends coaxially to the bellows structure. This coil spring can fulfill three advantageous functions: ensuring the basic straight guidance in the pushing normal, the easy guidance of lateral steering cables, which serve to limit expansion and to steer, and the prevention of excessive diameter expansion in the event of unintentional overpressure.

According to a preferred embodiment, a slightly stretchable helical spring is arranged inside the bellows element, which is surrounded by a pressure-tight and elastic film. This inner coil spring allows additional functional elements to be guided according to the catheter principle (probes, tools, etc.).

The pushing expansion at the pushing module occurs through the pressure or volume loading of the supply medium located in the bellows structure. In the opposite case, a relative negative pressure is created for contraction.

Preferably, a supply medium is used which is actuated by a pump and fed from a closed reservoir. When a negative pressure is generated, the supply medium is returned to the reservoir. The reservoir can be a piston-cylinder system, an elastic storage body or another microrobotic unit. When using external pressure, a pneumatic supply medium (e.g., nitrogen) is preferred due to the expected length of the hose system.

According to a particularly preferred embodiment, the microrobotic unit can be steered by generating the feed movement deviating from the central axis of the unit. This steering is preferably achieved by temporarily fixing the lateral extensibility of the bellows structure of the pushing module. For this purpose, steering elements, in particular steering cables or steering springs, are attached to the bellows structure and extend axially and can be temporarily blocked by clamping elements. To activate the steering, it is sufficient to clamp the steering cable or the steering spring. Due to the temporary prevention of lateral bellows expansion achieved in this way, significantly less energy is required than with active cable steering.

Particularly preferred for reliable steering are 2×2 antagonistic steering cables. The steering cables are preferably kept taut by an elastic element. As steering cable clamping mechanisms, frictional locking, positive locking, wedge and looping principles can be used, which are switched electrically and/or by a fluid.

In a modified embodiment, the steering elements are formed by activatable stiffening layers applied to the bellows structure. The stiffening layers can be activated, for example, by an electric current or by a change in temperature, i.e., their stiffness can be influenced in order to block or reduce the expansion of the bellows on one side.

The at least one front and one rear spreadable module each have the task of fixing the microrobotic unit in its position in the spread state and/or of creating a counter bearing when generating a feed or retraction force. The aim is to achieve an optimum of minimal internal force generation with maximum anchoring effect. It is understood that more than two spreadable modules can be provided on the unit, which complement each other and may further increase the flexibility of the unit.

According to a preferred embodiment, the surface structure of the elastic balloon envelope of the respective spreadable module is formed as a bristle-like structure. The bristles are sufficiently stiff to generate a high friction effect with the surrounding tissue when in the upright position (partial form fit), and on the other hand are sufficiently soft so as not to injure the surrounding tissue during a feed movement (no micro hooks). As an alternative to the bristles, a surface with a so-called kirigami structure (folds with incisions) can be used on the balloon envelope.

According to a modified embodiment, the surface structure of the elastic balloon envelope of the respective spreadable module is formed by a cascaded structure. In particular, bubbles attached to the balloon envelope can themselves have attached nubs. This cascading allows a large extension of the surface structure when the supply medium is fed in (pressure build-up) and at the same time provides a mechanical structure that ensures optimal contact with the surrounding tissue to maximize the holding force. On the other hand, when negative pressure is generated in the spreadable modules, the holding and friction effect on the surrounding structure is completely eliminated. The bubbles on the surface of the balloon envelope fall completely inwards and then lie below the base diameter of the spreadable module. The nubs are also placed inwards in a defined manner. Particularly preferably, four radial nubs are arranged around a central nub. Thus, the pushing resistance at the reduced-diameter spreadable module is reduced to a minimum. A further advantage of this embodiment is that the switchable movement inhibition acts independently of the direction of movement of the pushing module. In modified embodiments, the outward-facing tips of the nubs can be provided with additional movement-inhibiting structures (e.g., micro bristles, micro grains).

The expansion or contraction of the balloon envelope of the spreadable module occurs by generating overpressure or negative pressure (hydraulic or pneumatic) in the spreadable module. Preferably, the shape and material of the balloon envelope are selected so that it can be expanded (stretched) without its own resistance to movement (e.g., by overcoming intrinsic elasticities) and can be contracted to a minimum diameter by negative pressure in order to thus offer minimal resistance to movement during the feed process. The inflow and outflow of the supply medium is controlled via control elements, in particular switchable micro valves.

According to a preferred embodiment, the front spreadable module, in addition to anchoring or fixing in the tissue, also has the task of so-called blunt preparation of the connective tissue in the direction of movement. This function is supported by the mandrel arranged on the front spreadable module and/or by a special design of the balloon envelope of the front spreadable module.

### BRIEF DESCRIPTION OF THE DRAWINGS

Further advantages, details and modifications of the invention are described below on the basis of preferred embodiments, with reference to the drawing. Shown are:

FIG. 1 a perspective view of a first embodiment of a microrobotic unit according to the invention;

FIG. 2 an exploded perspective view of the microrobotic unit according to FIG. 1;

FIG. 3 side views of the microrobotic unit according to FIG. 1 in two different operating states;

FIG. 4 three images of a bellows structure of a pushing module;

FIG. 5 a schematic representation of a steering mechanism of the microrobotic unit;

FIG. 6 a basic representation of the microrobotic unit with a continuous central channel;

FIG. 7 an exploded perspective view of a second embodiment of the microrobotic unit;

FIG. 8 the embodiment according to FIG. 7 in two different feed states.

### DETAILED DESCRIPTION

FIG. 1 shows a simplified perspective view of a first embodiment of a microrobotic unit 01. The unit 01 has a front spreadable module 02 and a rear spreadable module 03, which are spaced apart from each other and (in the resting state) are axially aligned with each other. A pushing module 04 extends between the two spreadable modules 02, 03. On the rear spreadable module 03 there is a supply connection 05, to which an axially extending hose system 06 is connected. In the example shown, the hose system 06 comprises a central channel 06a and several additional channels 06b. At the front end of the unit 01 in the feed direction there is a truncated cone-shaped mandrel 07 with a front opening 08.

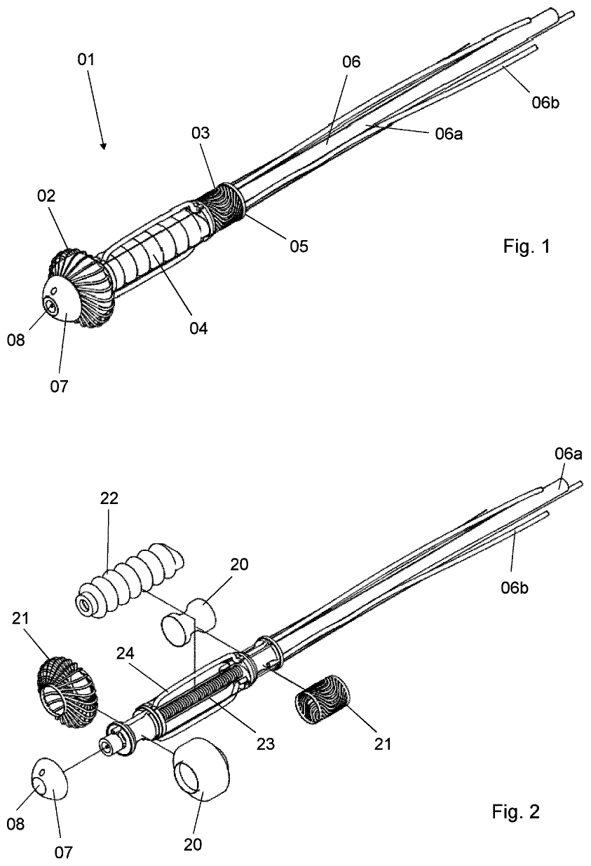

In FIG. 2, the microrobotic unit 01 is shown in an exploded view. It can be seen that at least the central channel 06a extends through the spreadable modules 02, 03 and the pushing module 04 and ends at the mandrel 07. In this way, for example, miniaturized tools can be guided to the front opening 08 in the mandrel 07 and can emerge from there. The additional channels 06b are routed to the respective module in order to supply it with a supply medium. Furthermore, control elements (not shown) are provided which control the supply and removal of the supply medium to the individual spreadable modules and to the pushing module as a function of control signals. These control elements can be integrated into the unit or positioned at a remote end of the hose system.

The two spreadable modules 02, 03 each have a balloon envelope 20, which can be expanded or contracted with the help of the supply medium. In FIGS. 1 and 2, the front spreadable module 02 and its balloon envelope are shown in the expanded state, while the rear spreadable module 03 with its balloon envelope is shown in the contracted state. The balloon envelopes 20 are each provided with a surface structure 21, which can be designed as an independent sleeve as shown in FIG. 2, or in other designs is designed integrally with the balloon envelope, for example in the form of bristles, nubs or cascaded structures integrated into the balloon envelope. In the expanded state, the surface structure 21 anchors itself in the surrounding tissue without damaging the tissue.

Furthermore, it can be seen from FIG. 2 that the pushing module 04 has a bellows structure 22. The central bellows element of the bellows structure can, for example, be made from a highly elastic silicone (e.g., using a 3D printing process). The dimensioning depends on the target functionality; for example, partial wall thickenings can be provided to simulate a grid structure. In this embodiment, an inner coil spring 23 runs inside the bellows 22. External supply channels 24 are provided on the outside.

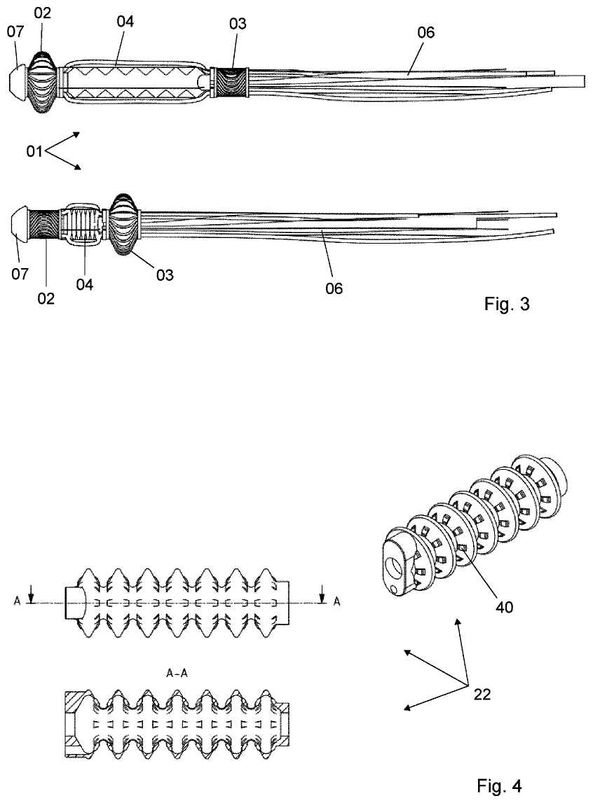

FIG. 3 shows the microrobotic unit 01 in two different operating states to illustrate the realization of a feed movement. In the upper figure, the front spreadable module 02 is expanded by the supply of the supply medium and thus has a significantly larger outer diameter than the pushing module 4. The pushing module 4 is also supplied with a supply medium so that it assumes a maximum expansion in the axial direction, while its outer diameter does not change significantly due to the support structure. The supply medium is withdrawn from the rear spreadable module 03 so that its balloon envelope 20 is contracted and assumes a reduced outer diameter (substantially corresponding to the diameter of the pushing module).

In the lower illustration of FIG. 3, the front spreadable module 02 is in the contracted state, i.e., the supply medium has been withdrawn from it. Instead, the rear spreadable module 03 is in the expanded state, i.e., it has been supplied with supply medium and its balloon envelope 20 has expanded in order to be fixed in the surrounding tissue. The pushing module 04 is axially contracted, i.e., the supply medium has been removed. To achieve this state, in chronological order, the axial length of the pushing module was first contracted, causing the rear spreadable module (in the contracted state) to move in the feed direction, as can be clearly seen from the comparison of the two images in FIG. 3. In the next step, the rear spreadable module 03 is extended to fix it and the front spreadable module 02 is contracted to prepare it for the next feed movement. This achieves the state shown in the bottom image. In the following step (not shown), the pushing module 04 is again supplied with supply medium so that it expands axially and pushes the front spreadable module 02 forward in the feed direction.

FIG. 4 shows a side view, a longitudinal sectional view and a perspective view of the bellows structure 22 according to a possible embodiment. It is essential for the bellows structure that its axial expansion changes due to the supply medium being added or removed (filling or emptying the bellows element). For example, several toggle levers arranged in a row can be integrated into the outer wall. In the embodiment shown in FIG. 4, a grid structure 40 is embedded in the silicone material of the bellows element. The outer diameter of the bellows system remains substantially constant when pressurized.

FIG. 5 shows in a sectional view the basic structure of the microrobotic unit 01 taking into account a steering mechanism. In general, the direction of the feed movement can be changed by keeping the axial extension on one axial side of the pushing module 04 smaller than on the opposite side. In the simplest case, at least two steering cables 50 are arranged as steering elements on opposite sides of the bellows element 22. Each steering cable 50 is assigned a clamping element 51, upon activation of which (e.g., by means of the supply medium) the steering cable can be fixed in a shortened state. As a result, the bellows element 22 on the side of the shortened steering cable can expand less axially, so that the feed movement is curved in this direction.

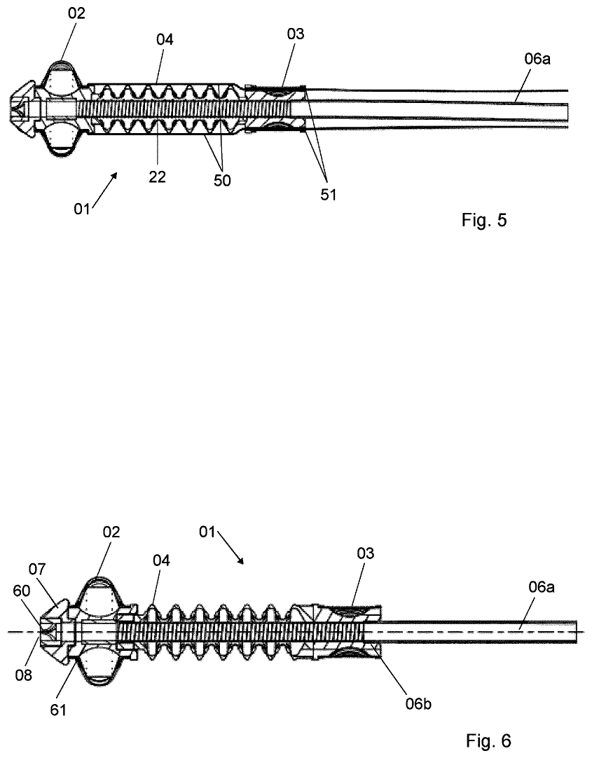

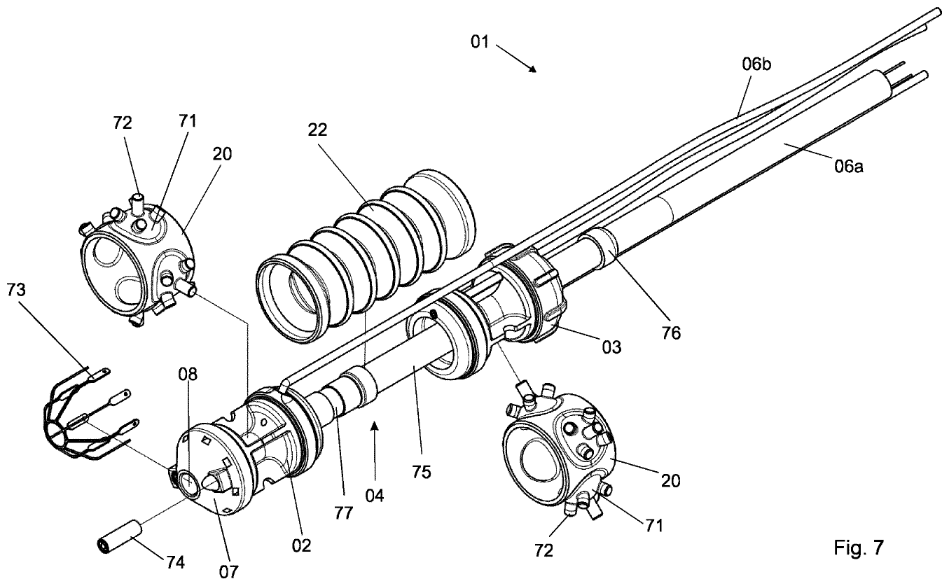

FIG. 6 shows in a sectional view again details of the microrobotic unit 01 according to the previously described embodiment. It is clearly visible here that the central channel 06a runs through all modules arranged axially in a row, up to the front opening 08 in the front mandrel 07. In the embodiment shown, the central channel 06a is closed at the front end by internal flaps 60, which on the one hand allow tools to be pulled out or infusions or the like to be administered, and on the other hand prevent the undesired entry of liquid or tissue. A locking mechanism 61 can also be arranged in the central channel 06a, for example to secure a surgical tool when it has been fed via the central channel. Lastly, it is evident that supply medium can be specifically supplied to or removed from the individual modules via the additional channels 06b in order to set different pressures there, so that a feed movement can be initiated in the manner described above.

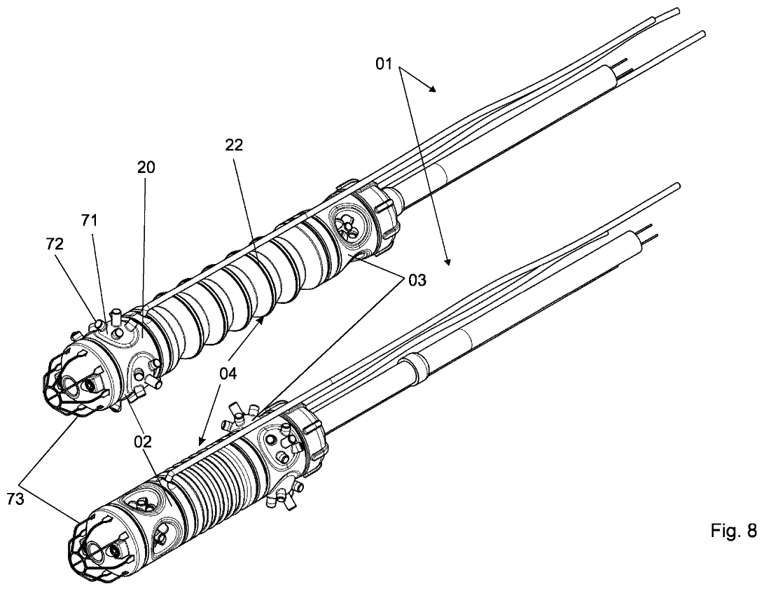

FIG. 7 shows a perspective exploded view of a second embodiment of the microrobotic unit 01. The front spreadable module 02 and the rear spreadable module 03 each have adapted balloon envelopes 20. In this embodiment, the balloon envelopes 20 have a plurality of cascaded surface structures, each consisting of a bubble-like dome 71 with a plurality of bristles 72 attached thereto. The plurality of domes and their associated bristles can expand radially outward when the supply medium is supplied, while other sections of the main body of the balloon envelope 20 can be rigid and have a fixed diameter. The surface structures formed by the domes and the bristles act hierarchically, i.e., when the pressure in the spreadable module increases, depending on the selected material stiffness, the domes 71 are initially expanded radially outwards, whereupon the bristles 72 on the domes 71 then straighten up or extend outwards when the pressure is further increased (or in the reverse order if the materials are selected differently). This hierarchical arrangement of several surface structures has the advantage that, upon subsequent pressure reduction, the bristles 72 fold into the dome 71 and are then pulled further inward with the dome, thus no longer protruding beyond the minimum diameter of the balloon envelope 20 and generating only minimal frictional resistance on the cavity walls during further forward movement of the unit 01.

FIG. 7 also shows a protective grid 73 which is mounted over the frustoconical mandrel 07 in order to protect it and the units mounted there. In this embodiment, this includes a micro camera 74, which is positioned in the mandrel 07.

In the embodiment shown in FIG. 7, a rigid or flexible central tube 75 runs centrally in the unit 01 and forms a section of the central channel 06a. At its end facing away from the front opening 08, the central tube 75 is guided through a smooth-running and pressure-tight bearing 76. When an axial movement of the unit is caused by the controlled pressure-negative-pressure application of the bellows structure 22, the rear spreadable body 03 slides on the central tube 75 and is guided by it. In the area of the bellows structure 22, the central tube 75 is preferably separated and connected to an elastic hose piece 77. Thus, the front end of the unit 01 with the front spreadable body 02 is flexibly movable relative to the following section. By operating the internal steering cables, the elastic hose piece 77 can be partially shortened in order to cause a change in direction of the front spreading body 03 with the mandrel 07 during the feed movement.

FIG. 8 shows the embodiment according to FIG. 7 in two different feed states in perspective view. In the upper illustration, the front spreadable module 02 in the axial direction is pressurized by the supply medium so that the dome 71 and the bristles 72 are radially expanded in order to fix the front spreadable module in the surrounding tissue. The pushing module 04 is supplied with supply medium so that the bellows structure 22 is expanded axially without experiencing a significant change in cross-section in the radial direction. The rear spreadable module 03 is not subjected to overpressure or is preferably connected to a negative pressure so that its surface structure is minimized in the radial direction, i.e., the dome and the bristles do not protrude beyond the minimum outer diameter of the balloon envelope of the rear spreadable module. In order to get from the state shown in the upper image to the state shown below, the supply medium is first pumped out of the bellows structure 22 so that it contracts axially, whereby the rear spreadable module 03 is displaced axially forward.

In the lower image of FIG. 8, the front spreadable module 02 is not pressurized or is preferably connected to negative pressure. The dome 71 and the bristles 72 are retracted radially. Thus, the front spreadable module 02 assumes its minimum cross-section. The pushing module 04 is contracted in the axial direction, either by integral spring forces and/or by reducing the pressure of the supply medium. The rear spreadable module 03 is subjected to pressure so that the dome and bristles are moved radially outwards in order to fix the rear spreadable module in the surrounding tissue. When supply medium is supplied to the pushing module 04 in the next step, the bellows structure 22 of the pushing module 04 is extended so that the front spreadable module 02 is pushed axially forward.

1. A microrobotic unit for propulsion movement and positioning in organic cavities, comprising:
a front spreadable module and a rear spreadable module,
each spreadable module having at least one outer balloon envelope the volume of which expands at least partially when the supply medium is supplied, a surface structure being provided on the outside of the balloon envelope and, in the expanded state, the balloon envelope to be anchored in the surrounding tissue;
a pushing module which is arranged between the two spreadable modules and connected to them, wherein
he pushing module has a bellows structure which causes an axial expansion of the pushing module when the supply medium is supplied and an axial contraction of the pushing module when the supply medium is removed, wherein the bellows structure has an outer, axially extending support structure to prevent or limit a change in its dimensions in the radial direction;
a supply connection which is axially connected to the rear spreadable module and serves to supply at least one supply medium; wherein the surface structure of the balloon envelope has a plurality of domes which can be expanded in the radial direction of the balloon envelope and on which bristles are formed which can also be expanded in the radial direction of the balloon envelope;
the axially extending support structure of the bellows structure is shaped like a plurality of toggle lever structures arranged in a row.

2. The microrobotic unit according to claim 1, wherein central channel extends through the rear spreadable module and the pushing module to the front spreadable module.

3. The microrobotic unit according to claim 1, wherein a frustoconical mandrel having a central passage opening is mounted axially in front of the front spreadable module.

4. The microrobotic unit according to claim 1, further comprises a hose system which is coupled to the supply connection.

5. The microrobotic unit according to claim 1, further comprising one or more further modules which are arranged axially behind the rear spreadable module, wherein the further modules in particular comprise storage containers, battery units and/or data processing components.

6. The microrobotic unit according to claim 1, wherein one or more sensors which detect environmental parameters and/or state parameters of the unit.

7. The microrobotic unit according to claim 1, wherein the bellows structure is surrounded by an outer coil spring or a Nitinol structure.

8. The microrobotic unit according to claim 1, wherein an inner coil spring with an elastic sheath is arranged in the interior of the bellows structure.

9. The microrobotic unit according to claim 1, wherein steering elements run laterally on the bellows structure and can be activated to direct the feed direction of the unit.

10. (canceled)

11. (canceled)

12. The microrobotic unit according to claim 2, wherein a flap mechanism for selectively closing and opening the central channel is arranged at the front end of the central channel.

13. The microrobotic unit according to claim 12, characterized in that in the central channel there is arranged a locking mechanism, with which instruments guided in the central channel can be fixed.

14. The microrobotic unit according to claim 1, wherein the bellows structure has a plurality of toggle lever elements arranged in a row in its outer wall.

15. The microrobotic unit according to one claim 1, wherein the bellows structure has a structure made of a shape memory alloy or of plastic on or in its outer wall.
